(19) United States
(12) Patent Application Publication
(10) Pub. No.: US 2025/0029717 A1
(43) Pub. Date: Jan. 23, 2025

(54) VISUALIZATION OF MACHINE LOG DATA USING AN ORGANIZATIONAL SUBTREE

(71) Applicant: KONINKLIJKE PHILIPS N.V., EINDHOVEN (NL)

(72) Inventors: Johannes Henricus Maria KORST, EINDHOVEN (NL); Serverius Petrus Paulus PRONK, VUGHT (NL); Mauro BARBIERI, EINDHOVEN (NL); Marc André PETERS, VELDHOVEN (NL)

(21) Appl. No.: 18/711,173

(22) PCT Filed: Nov. 30, 2022
(86) PCT No.: PCT/EP2022/083751

§ 371 Date: May 17, 2024

Related U.S. Application Data
(60) Provisional application No. 63/285,514, filed on Dec. 3, 2021.

(51) Int. Cl. G16H 40/40

(52) U.S. Cl. CPC G16H 40/40

(57) ABSTRACT

A non-transitory computer readable medium (107, 127) stores instructions readable and executable by at least one electronic processor (101, 113) to perform a method (200) of selecting a subset of log data (130) automatically generated by a medical device (120) undergoing servicing. The method includes generating chains (134) of a hierarchical components chart (132) representing components of the medical device (120), wherein each chain begins at a node (136) of the hierarchical components chart representing a component of the medical device involved in the servicing and extends upward through the hierarchy of the hierarchical components chart: identifying a subset of components of the medical device corresponding to the nodes of the generated chains: selecting the subset of the log data as messages (138) of the log data associated with the identified subset of components of the medical device; and displaying at least a portion of the subset of the log data.

### FIELD

The following relates generally to the medical device maintenance arts, medical imaging device maintenance arts, medical device maintenance visualization arts, and related arts.

### BACKGROUND

Medical devices and some other complex computerized devices (for example, commercial airliners, industrial robots, or so forth) produce warning and error messages that are logged and sent to a central storage facility to allow inspection by a remote service engineer, whenever an issue has been reported by the user of the device. With each warning and error message a unit and subunit (and possibly sub-sub-units, and so forth) can be specified. This information simplifies determining the root cause. If the unit is inspected and subunit information of the warning and error messages that are generated in a time interval prior to the moment in time that the issue is reported by the user, then this may help identifying the root cause of the issue.

However, numerous warning and error messages are typically generated prior to the moment that an issue is reported. These messages can have different associated unit and subunit information. It can be difficult for a service engineer to comprehend which log data messages are relevant to a current servicing task. Moreover, a thorough understanding of the import of the log messages may require recognition of interrelationships between the messages. For example, a malfunction of a first sub-unit may generate a first message, and that malfunction may in turn result in further log messages generated by the unit containing that subunit, and/or other sub-units of that unit. Recognition of this sequence of messages may be useful for diagnosing the root cause (in this case, the malfunction of the first sub-unit).

The following discloses certain improvements to overcome these problems and others.

### SUMMARY

In one aspect, a non-transitory computer readable medium stores instructions readable and executable by at least one electronic processor to perform a method of selecting a subset of log data automatically generated by a medical device undergoing servicing. The method includes generating chains of a hierarchical components chart representing components of the medical device, wherein each chain begins at a node of the hierarchical components chart representing a component of the medical device involved in the servicing and extends upward through the hierarchy of the hierarchical components chart; identifying a subset of components of the medical device corresponding to the nodes of the generated chains; and selecting the subset of the log data as messages of the log data associated with the identified subset of components of the medical device; and displaying at least a portion of the subset of the log data.

In another aspect, a non-transitory computer readable medium stores instructions readable and executable by at least one electronic processor to perform a method of selecting a subset of log data automatically generated by a medical device undergoing servicing. The method includes receiving, via a user input device, a selection of one or more nodes of a sub-chart displayed on a display device; and displaying a messages of the subset of the log data associated with the component or components corresponding to the selected one or more nodes.

In another aspect, a method of assisting with servicing of a medical device includes generating chains of a hierarchical components chart representing components of the medical device, wherein each chain begins at a node of the hierarchical components chart representing a component of the medical device involved in the servicing and extends upward through the hierarchy of the hierarchical components chart; merging the generated chains to form a sub-chart of the hierarchical components chart, wherein the highest node in the hierarchy that is included in the sub-chart is (i) a root node of the hierarchical components chart representing the medical device or (ii) a highest common node of the hierarchical components chart that is common to all of the generated chains; and displaying, on a display device, the sub-chart.

One advantage resides in organizing a chart of medical device components according to priority of the components of the medical device.

Another advantage resides in reducing an amount of an organizational chart of medical device components for visualization to a user.

Another advantage resides in reducing time spent learning about components of a medical device that irrelevant to a current servicing session of the medical device.

Another advantage resides in expediting a time spent during a servicing session of a medical device.

Another advantage resides in providing improved visualization of an organizational chart in the context of a task or scenario involving only a subset of the nodes of the chart.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for servicing medical device in accordance with the present disclosure.

FIG. 2 shows exemplary flow chart operations of the system of FIG. 1.

FIGS. 3-6 show example outputs of the system of FIG. 1.

### DETAILED DESCRIPTION

Embodiments disclosed herein repurpose an electronic organizational chart (also known as an organigram) of a type sometimes used in representing the organizational hierarchy of employees (or workers or other human resources) of a corporation, governmental agency, non-profit group, or other type of hierarchical organization. The repurposing represents interrelationships of log data messages generated by a medical device or other complex computerized device or system using an analogous hierarchical chart. In this repurposing, nodes of a hierarchical components chart (analogous to an organizational chart of a hierarchical organization) represent units, sub-units, sub-sub-units, and so forth of the medical device. This approach recognizes that units of the medical device (possibly referred to by similar nomenclature such as components), sub-units (or sub-components, etc.), sub-sub-units (or sub-sub-components, etc.), and so forth naturally form a hierarchy. For example, if a medical device includes units A, B, C . . . ; and unit A includes sub-units A1, A2, A3 . . . ; and sub-unit A1 includes sub-sub-units A1.1, A1.2, A1.3 . . . , and so forth; and units B, C . . . are similarly decomposed into sub-units, sub-sub-units and so forth; then a hierarchy can be defined, which is referred to herein as a hierarchical components chart. In this chart, the medical device is the root node; units A, B, C . . . are the first level nodes of the chart directly below the root node; sub-units A1, A2, A3 . . . are the second level nodes underneath the node representing unit A; sub-sub-units A1.1, A1.2, A1.3 . . . are third level nodes underneath the node representing sub-unit A1; and so forth; and likewise for branches of the hierarchical components chart branching off nodes representing units B, C. . . . The resulting hierarchical components chart can be directly analogized to the organization chart of employees of a corporation or other organization. Furthermore, messages of log data, such as error messages and/or warning messages, generated by the medical device can be assigned to nodes of the hierarchical components chart corresponding to the units, sub-units, sub-sub-units, etc. associated with those messages. The hierarchical components chart can thus provide a convenient visualization of relationships between the various messages of log data, so as to simplify their interpretation by a service engineer.

In general, the hierarchical components chart as described above can be used for visualization of warning and error messages of log data generated in a chosen time interval (e.g., a time interval around the time that a malfunction of the medical device was recognized). Such visualization using the hierarchical components chart can use visualization approaches analogous to those used in visualizing portions of an organigram of a corporation or other organization.

However, as further recognized herein, existing approaches for visualizing portions of an organigram have certain deficiencies. For example, consider visualization approaches for visualizing organizational relationships of meeting participants in a video call are conducted by a large organization. Such organizations typically have an organizational chart (also known as an organigram) with, for example, the CEO at the top of the organization hierarchy (hence, the root node), various vice presidents at the next level down, and so forth. In a large organization, different meeting participants may be from different departments, and so it would be useful for a meeting participant to quickly know the organizational posture of other meeting participants. In principle, the participant could bring up the organigram, however, as that may include thousands of persons, this is not an efficient solution. The analogous situation when using the disclosed hierarchical components chart for visualizing error and warning messages of log data in a time interval around a recognized malfunction, these messages may pertain to a small fraction of the total number of units, sub-units, sub-sub-units, etc. of the hierarchical components chart. Consequently, viewing the hierarchical components chart as a whole may not be very useful to the service engineer. In the analogous situation of a meeting, viewing the entire organigram may not be very useful for a meeting participant, since the meeting participants may only correspond to a small fraction of the total nodes of the organigram.

It is still further recognized herein that in such situations, it is likely that the nodes corresponding to units/sub-units/sub-sub-units/ . . . generating error and warning messages (or the nodes corresponding to meeting participants) will typically be drawn from a relatively localized branch or set of branches of the hierarchical components chart or organigram. In the case of a malfunction of a medical device, the warning and error messages are likely to mostly or entirely be drawn from sub-units (or sub-sub-units, etc.) within a branch of the hierarchical components chart corresponding to a single unit, or at most within branches representing two or a few interacting units. Analogously, the meeting participants of a meeting are likely to mostly or entirely be drawn from a single department or at most a two or three collaborating departments. Based on these insights, improved visualization of the nodes representing error and warning messages of log data from around the time of a malfunction (or analogously, of the nodes representing meeting participants) is disclosed as follows.

The hierarchical components chart or organigram can be filtered by generating a chain for each node generating a warning or error message (or for each meeting participant) running upward in the hierarchy of the hierarchical components chart or organigram from that node to the root (representing the medical device or CEO, for instance). This results in a set of chains that can be combined to form a sub-tree of the hierarchical components chart or organigram representing the hierarchical postures of all units/sub-units/sub-sub-units etc., or meeting participants, without the additional information of the entire hierarchical components chart or organigram.

In a variant embodiment of this visualization approach, the top of the resulting sub-tree is pruned by removing the common highest level(s), so that the sub-tree starts at the lowest-ranking person who is a manager of all meeting participants. For example, if the tree has a CEO, vice-presidents' level, and department heads level, and all meeting participants (indirectly) report to the same department head, then that department head serves as the root of the sub-tree with the higher-level vice-president and CEO pruned away. This approach recognizes that those upper levels of the organigram are unlikely to be of interest to the meeting participants. For example, if the organigram has a hierarchy of: CEO, business units, and departments within the business units, and the meeting participants are all within a single department, then it is not necessary to visualize the upper CEO and business unit portion of the organigram to provide meeting participants with an understanding of the organizational postures of other meeting participants. Similarly, in the case of visualizing error or warning messages of log data, the lowest-level common node is likely to represent the “region” of the medical device implicated in the malfunction, so that higher-level nodes above that lowest-level common node are unlikely to be of interest to the service engineer diagnosing the root cause of the malfunction.

To further illustrate the analogy of the hierarchical components chart to an organigram and use of the hierarchical components chart in diagnosing the root cause of a malfunction of a medical device (or other complex computerized device), following example is described. In this example, if the medical device is a magnetic resonance imaging (MRI) scanner then the “CEO” is the MRI scanner, the “vice presidents” are the various main units of the MRI scanner such as the patient support, magnet, gradient coils, RF system, et cetera, the “department heads” may be various subsystems of these main units, and so forth until the leaves of the tree are reached which represent scanner parts that cannot be further broken down (that is, scanner parts that are replaced as whole units).

In use, a time interval of log data comprising messages are downloaded during servicing. Typically, the time interval is located around the time the malfunction being diagnosed was recognized, e.g. an hour or more before the malfunction was recognized extending to perhaps a short time thereafter. Each message is associated to a node in the medical device tree, and a sub-tree can be created analogously to the sub-tree of meeting participants. The messages are annotated to the nodes of this sub-tree, which can then be displayed and/or manipulated by the service engineer (SE). For example, the SE may turn on or off various nodes of the sub-tree, e.g. if the SE believes the problem relates to the RF system, then the other nodes can be turned off so that only messages annotated to nodes of the RF system sub-tree are retained.

In some embodiments, the sub-tree is not constructed with all messages, but only with messages that are deemed relevant. For example, routine and repetitive messages such as “table moving” that occur during every scan might not be included in the sub-tree. In one approach, this initial pre-filtering of the messages may remove all messages other than “abnormal” messages such as error messages and warning messages.

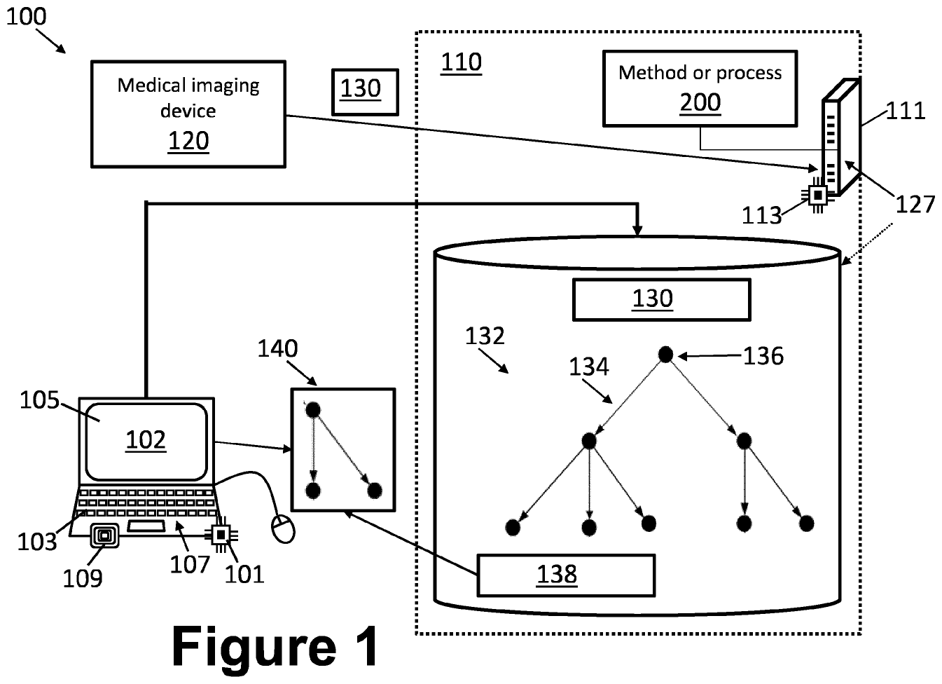

With reference to FIG. 1, an illustrative servicing support system 100 for supporting a service engineer in servicing an electronic device 120 (e.g., a medical imaging device —also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. (More generally, the disclosed approach can be applied in conjunction with any type of computerized device that automatically generates log data, e.g., the approach could be applied to a commercial airliner, radiation therapy device, or so forth). As shown in FIG. 1, the servicing support system 100 includes, or is accessible by, a service device 102 that may for example be a workstation or electronic processing device used by a user (e.g., a service engineer (SE), such as a remote SE (RSE) or a field SE (FSE)). The service device 102 may for example be a portable device such as a notebook computer that is carried or accessed by an RSE. The service device 102 can be a desktop computer or a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g., notebook computer, tablet computer, or so forth) carried by an RSE performing diagnosis of a fault with the imaging device and ordering of parts. In another example, the service device 102 may be the controller computer of the imaging device under service, or a computer based at the hospital. In other embodiments, the service device may be a mobile device such as a cellular telephone (cellphone) or tablet computer.

The service device 102 includes a display device 105 via which alerts generated by predictive failure models are displayed, along with likely root cause and service action recommendation information as disclosed herein. The service device 102 also preferably allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 for interfacing with the servicing support system 100. The service device 102 also includes a communication interface 109 to communicate with a backend server or processing device 111, which typically implements the computational aspects of the servicing support system 100 (e.g., the server 111 has the processing power for implementing computationally complex aspects of the servicing support system 100). Such communication interfaces 109 include, for example, a wired and/or wireless Ethernet interface (e.g., in the case in which the service device 102 is an RSE workstation); or in the case in which the service device 102 is a portable FSE device the interface 109 may be a wireless Wi-Fi or 4G/5G interface or the like for connection to the Internet and/or an intranet. Some aspects of the servicing support system 100 may also be implemented by cloud processing or other remote processing (that is, the server computer 111 may be embodied as a cloud-based computing resource comprising a plurality of interconnected servers).

In illustrative FIG. 1, the servicing support system further includes a backend 110 (e.g., implemented and/or owned by the imaging device vendor or leased by the vendor from a cloud computing service provider). The backend 110 receives log data (e.g., a machine log automatically generated by the medical imaging device 120, a service log for the medical imaging device 120, and/or so forth) on a continuous or occasional basis (e.g., in some setups the imaging device 120 uploads machine log entries to the backend 110 on a daily basis). The backend processing for performing predictive fault modeling and (as disclosed herein) maintenance service analyses is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single medical imaging device 120, more generally the database backend 110 will receive log data from many medical imaging devices (e.g., tens, hundreds, or more imaging devices) and performs the disclosed processing for a medical imaging device undergoing servicing using the log data generated by that device.

The non-transitory computer readable medium 127 stores log data 130 received from the medical device 120. The non-transitory storage medium 127 also stores instructions executable by the electronic processor 113 of the backend server 111 to perform a method 200 of selecting a subset of the log data 130 automatically generated by the medical device 120 while undergoing servicing.

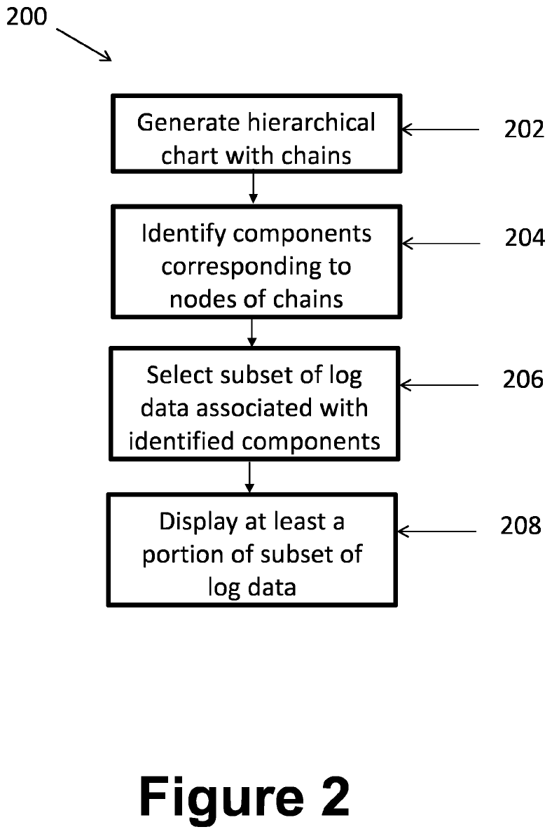

With continuing reference to FIG. 1 and further reference to FIG. 2, an illustrative embodiment of the method 200 executable by the electronic processor 113 of the backend server 111 is diagrammatically shown as a flowchart. In some examples, the method 200 may be performed at least in part by cloud processing.

At an operation 202, a hierarchical components chart 132 with one or more chains 134 is generated. For each message (after optional pre-filtering, for example to exclude messages that are not warning or error messages in one example), the message is associated to a node of the hierarchical components chart 132 corresponding to a specific unit, sub-unit, sub-sub-unit, or so forth to which the message relates. Typically, this can be done based on the content of the message that identifies the unit, sub-unit, sub-sub-unit, or so forth that generated the message, or it is the topic of the message. A chain through the hierarchical components chart 132 is then created starting at that node and moving upward in the hierarchy to the root node (which represents the medical device). Each chain 134 thus begins at a node 136 of the hierarchical components chart 132 representing the component of the medical device 120 (e.g., a medical imaging device) to which the error message is associated and extends upward through the hierarchy of the hierarchical components chart 132 to the root node. As used herein, “extends upward” refers to moving upward to successively higher levels of the hierarchical components chart 132. In some examples, each chain extends 134 upward through the hierarchy of the hierarchical components chart 132 to a root node 136 of the hierarchical components chart 132. That is, each chain 134 extends upward through the hierarchy of the hierarchical components chart 132 to the root node. Optionally, after all the chains are created, a lowest-level common node 136 of the hierarchical components chart 132 that is common to all of the generated chains 134 is identified, and any nodes above that lowest-level common node are removed from the chains. By way of illustration, if all the chains include a single common sub-unit, then they necessarily also include a single common unit node (namely the unit to which the common sub-unit belongs) and also necessarily include the root node (corresponding to the medical device). hence, the common unit node and the root node can be removed from all the chains.

At an operation 204, a subset of components of the medical device 120 corresponding to the nodes 136 of the generated chains 134 are identified. At an operation 206, a subset of the log data 130 are selected as messages 138 (e.g., error messages, warning messages, and so forth) of the log data 130 associated with the identified subset of components of the medical device 120.

At an operation 208, at least a portion of the subset of the log data 130 is displayed on the display device 105 of the electronic processing device 102. To do so, the generated chains 134 are merged together to form a sub-chart 140 of the hierarchical components chart 132, and the sub-chart 140 is displayed on the display device 105.

Based on the disclosure herein, it will be apparent that the method 200 is also readily adapted to provide visualization of meeting participants of a meeting. In this adaptation, an operation analogous to the operation 202 starts with nodes of the organigram representing meeting participants, and generates a chain for each meeting participant node extending upward through the organigram to the root node. Optionally, a lowest-level common node that is common to all the chains is identified and any nodes above that lowest-level common node are stripped from the chains. In an operation analogous to operations 204 and 206, information about the individuals or entities of the organigram represented by the nodes of the chains is collected (e.g. from data annotating the nodes of the organigram). In an operation analogous to the operation 202, the chains are merged together to form a sub-chart of the organigram that compactly visualizes the interrelationships of the meeting participants and their supervisors in the organigram hierarchy.

Returning to the illustrative application of visualizing log data messages, in some embodiments, the SE using the service device 102 can use the user input device 103 to select one or more of the nodes 136 of the displayed sub-chart 140, and the messages 138 of the subset of the log data 130 associated with the component or components corresponding to the selected one or more nodes 136 can be displayed on the display device 105. In another example, when one or more of the nodes 136 are selected, the subset of the log data 130 are updated by removing the messages 138 of the subset of the log data 130 associated with the component or components corresponding to the selected one or more nodes 136 from the subset. In yet another example, when one or more of the nodes 136 are selected, the subset of the log data 130 are updated by only displaying the messages 138 of the subset of the log data 130 associated with the component or components corresponding to the selected one or more nodes 136 or to any descendants of these one or more nodes 136 present in the hierarchy.

In other embodiments, the nodes 136 of the displayed sub-chart 140 can be annotated with messages 138 of the subset of the log data 130 associated with the corresponding components. To do so, a determination is made as to whether each message 138 of the subset satisfies a predetermined relevance criterion, and the nodes 136 of the displayed sub-chart 140 are annotated with only those messages 138 that satisfy the predetermined relevance criterion.

In further embodiments, the error and warning messages 138 generated by the medical device 120 are generated at a certain time and can include a timestamp, indicating their time of generation. This timestamp information could be used to generate a video clip that shows, in the sub-chart 140, the sequence of messages 138 that have been generated prior to the moment that the user reported the issue. The SE could then interactively view the sequence of events, as well as where they originated from.

### EXAMPLES

The following describes the system 100 and the method 200 in more detail. A rooted tree T (e.g., the hierarchical components chart 132) is constructed for a given meeting m a connected subtree T (m) ¿ T that only contains the participants of m and (some of) their managers. Upon request of one of the participants, by e.g. pressing a special button that is added to the digital meeting request, the subtree will be visualized in a pop-up window, that may show additional information when, e.g., hoovering over the different participants. By using the visualized subtree, less time needs to be spent on explaining details on the relative positioning of participants in the organizational chart, saving time for all participants.

The following examples pertain to embodiments of visualizing meeting participants using an organigram. As will be readily appreciated based on the disclosure herein, these approaches are readily adapted to visualizing log messages using the hierarchical components chart 132. The organizational chart can be defined by a rooted tree T=(V, E), where V denotes the set of vertices and E the set of edges. Each vertex ν∈V represents an employee, where root r represents the CEO of the organization. Each edge e=(ν, w)∈E indicates that ν is the direct manager of w. By definition of a tree, for each vertex ν∈V\{r} there is a unique path (e₁, e₂, . . . , eₘ) with m≥1 and e₁=(r, ν₁) and eᵢ=(νᵢ, νᵢ₊₁) for i=1, . . . , m−1, and, if m=1, ν₁=ν, and if m>1, eₘ=(νₘ, ν). All edges on the path from r to ν are assumed to be directed in the direction of ν.

For each edge(ν, w)∈E, let ν be the parent of w and w be a child of ν, and let C(ν) denote the set of children of ν, and let p(ν) denote the parent of ν. Furthermore, let A(ν) denote the ancestors of ν, being all the persons on the path from root r to vertex ν, including r and excluding ν. Likewise, let D(ν) denote all descendants of ν, i.e., the vertices that can be reached from vertex ν using the edges in E, excluding ν. Root r is positioned at level 1, the children of r at level 2, the children of the children of r at level 3, et cetera. The level of vertex ν is denoted by l(ν).

In addition, each vertex ν is given a unique identifier I(ν) being a sequence of integers of length l(ν). C(ν), the children of ν, are successively numbered as 1, 2, . . . using some arbitrary but fixed order. In this way, root r can be identified by sequence (1), the second child of r by (1, 2). The fourth child of this second child of r by (1, 2, 4), et cetera.

For a given meeting m, let P(m) denote the persons that are invited to participate in meeting m. Let V(m) denote the vertices in T that correspond to these persons. Now, the subtree T(m)=(V′(m), E′(m)) is constructed as follows.

Let Iₘᵢₙ(m)=min {l(ν)|ν∈V(m)} and max(m)=max {l(ν)|ν∈V(m)} denote the smallest and largest levels that occurs in V (m).

V(m) can be partitioned into subsets Vₚ(m), Vₚ₊₁(m), . . . , V_{q}(m), where p=lₘᵢₙ and q=\max, such that Vᵢ(m) contains the vertices ν with l(ν)=i.

The vertices in Vᵢ(m) are iteratively considered starting with the largest level, then the one but largest level, et cetera, as follows.

For each ν E Vᵢ(m), ν is added to V′(m), the vertex set of the subtree, and add parent p(ν) to Vᵢ₋₁(m), whenever it exists. Additionally, when p(ν) exists, (p(ν), ν) is added to E′(m) the edge set of the subtree. If Vᵢ₋₁(m) does not yet exist, it is added as additional subset.

The construction of the subtree will stop when V₁(m) is reached and root r is added to the subtree. In that case, the subtree will always contain root r and all the edges and vertices that lie on the paths from root r to the meeting participants are contained in the subtree.

Alternatively, construction of the subtree can be stopped at the point where Vᵢ(m) contains only one vertex. Let this single vertex be denoted by w. In this case, for each ν∈V(m), it holds that ν∈D(w). In this case, w is added to V′(m), but p(w) is not added to Vᵢ₋₁(m), and is (p(w),w) is not added to E′(m). In other words, in this case, the subtree contains the earliest common ancestor eca of the meeting participants and all edges and vertices that lie on the paths from eca to the meeting participants. The path from r to eca is not shown.

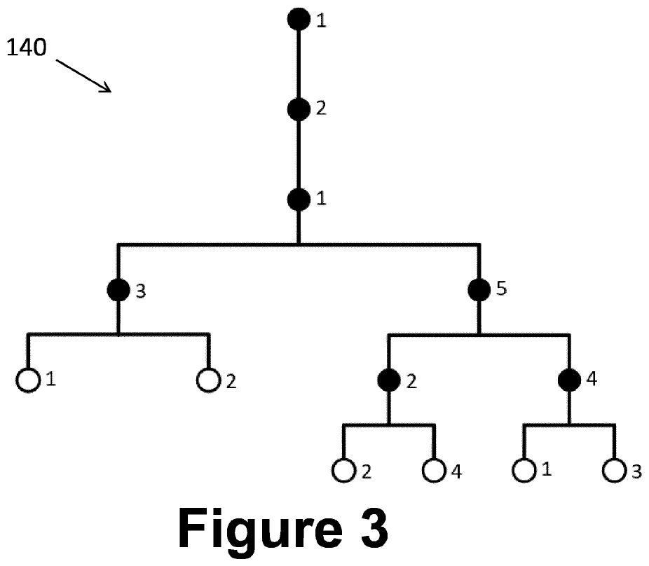

With reference next to FIGS. 3-6, some nonlimiting illustrative examples of suitable visualizations of the sub-tree of the hierarchical components chart 132 (or of the organigram in the case of visualizing meeting participants) are described. The examples of FIGS. 3-6 specifically refer to meeting participants visualized using a sub-tree extracted from an organigram, but can alternatively be applied to representing the nodes of sub-tree extracted from the hierarchical components chart 132 using the method 200 of FIG. 2 in the context of the system of FIG. 1.

FIG. 3 shows an example of a sub-chart 140 or sub-tree. If a meeting m has 6 participants, the participants can be identified by their respective paths, given by (1, 2, 1, 3, 1), (1, 2, 1, 3, 2), (1, 2, 1, 5, 2, 2), (1, 2, 1, 5, 2, 4), (1, 2, 1, 5, 4, 1), and (1, 2, 1, 5, 4, 3). The subtree containing root r can now be visualized as shown in FIG. 3. The names of the respective persons will be shown, instead of the numbers extracted from their identifiers.

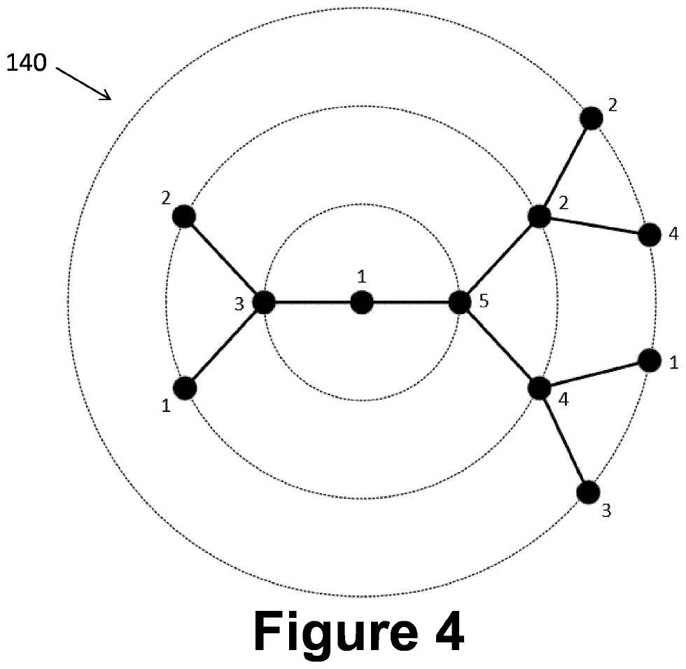

FIG. 4 shows another example of a sub-chart 140 or sub-tree, in which the sub-tree stops at the earliest common ancestor. Instead of placing the persons from the same level on the same horizontal line, these are now placed on the same circle. In the middle the earliest common ancestor is shown. Also here, the names of the persons will be shown, instead of their identifiers. The path from the earliest common ancestor to the root could have been added as a vertical line, where the vertices between the root and the earliest common ancestor are deliberately not positioned on the circles on which other vertices are already positioned, in order to make clear that they do not belong to the level that relates to these circles.

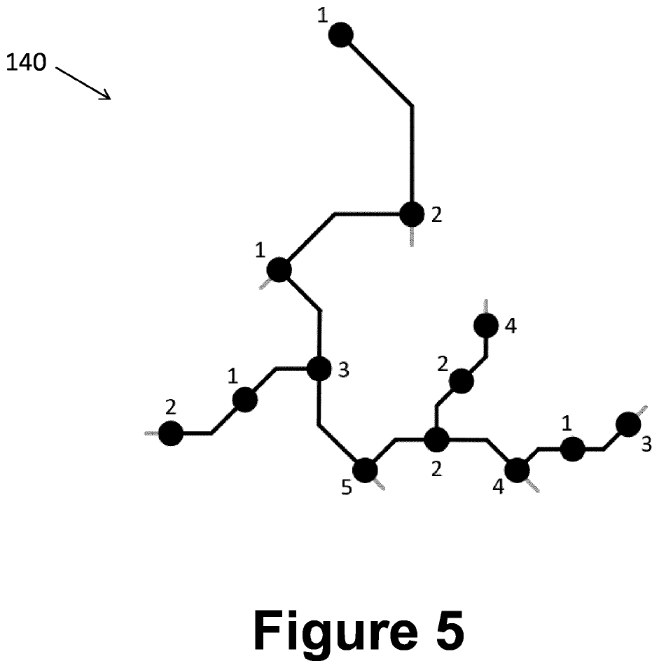

FIG. 5 shows another example of a sub-chart 140 or sub-tree that starts at the root and uses only horizontal, vertical, and diagonal line segments connecting a parent to its first child, or connecting a child to its next sibling. For example, the vertex with number 3 has three edges: an edge from its parent to itself, an edge from itself to its next sibling, numbered 5, and an edge to its own first child numbered 1. Each edge is drawn with two line segment of equal length, as if the edge bends halfway with plus or minus 45 degrees. In this way, each parent with its children forms a continuous curve with 45 degree bends between the successive vertices. The last child on this curve is given a small extending line segment to indicate that there can be more subsequent children, here shown in grey. The root is the only vertex without this extending line segment. This visualization can be beneficial in drawing relatively large trees. In this way all vertices of the same level are drawn on the same horizontal line or on the same circle can be avoided. This visualization is considered to be a more effective use of the available screen space.

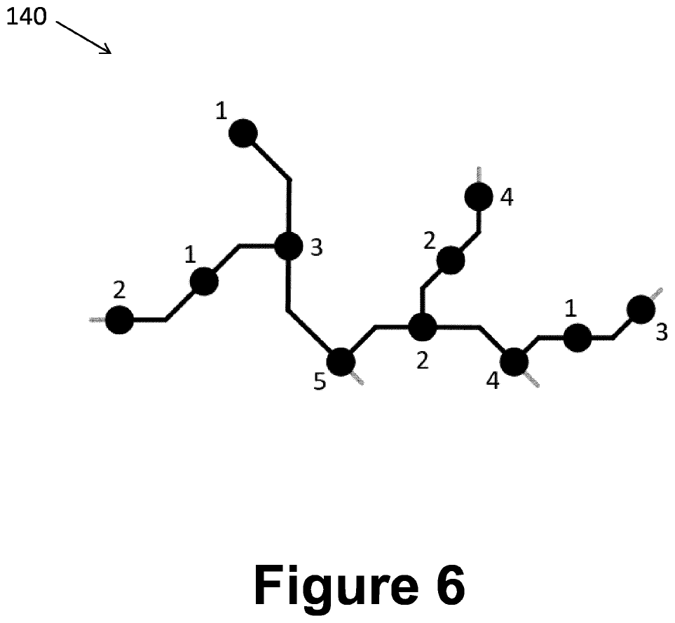

FIG. 6 shows another example of a sub-chart 140 or sub-tree that includes drawing the tree starting at the earliest common ancestor. It will be clear that these visualizations are just examples to illustrate the basic idea.

Additional information can be shown in the given subtree. In addition to the name of the persons, their functions or roles or the name of the units or departments to which these persons belong can be shown. Furthermore, the edges or their background could be given a specific colour indicating a specific part of the organization.

In addition, selecting a vertex ν may offer the user easy access to documents of which the authors have a large overlap with the descendants D(ν) of vertex ν. A list of these documents may be shown in a pop-up window, where the documents may be ordered appropriately considering the degree of overlap with the descendants D(ν) and the age of the documents.

For meetings that also have participants from outside the organization, one can easily arrange that the subtree visualization is only visible to persons inside the organization, for confidentiality reasons. In that case the button that triggers the visualization can simply be removed from the user interface for external participants.

An alternative application of the above basic idea is in reactive maintenance of medical devices.

With returning reference to the application of visualizing error messages of log data of a medical device (or other computerized system that generates log data), e.g., as described herein with reference to FIGS. 1 and 2, several different warning and error messages are typically generated prior to the moment that an issue is reported. These messages can have different associated unit and subunit information. For a service engineer to get a good overview of the messages that are logged in the time interval prior to the start of the issue, a rooted tree can be generated, containing all unit-subunit-message paths. This gives a good overview of the involved messages and how they relate to the different units and subunits of the device.

The tree can be visualized in an interactive setting, where the service engineer can decide only to visualize the error messages or both the warning and error messages. In addition, the relative frequency of each of the messages can be used to visualize only the messages that occur relatively frequent. The relative frequency can be based on how often a message is generated per time unit. This can be calculated over the whole installed base of similar devices or calculated for the specific device at hand.

It will be clear that this interactive visualization can be beneficial to quickly determine the root cause of an issue and help to avoid unplanned downtime.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
instructions readable and executable by at least one electronic processor to perform a method of selecting a subset of log data automatically generated by a medical device undergoing servicing, the method comprising:
generating chains of a hierarchical components chart representing components of the medical device, wherein each chain begins at a node of the hierarchical components chart representing a component of the medical device involved in the servicing and extends upward through the hierarchy of the hierarchical components chart;
identifying a subset of components of the medical device corresponding to the nodes of the generated chains;
selecting the subset of the log data as messages of the log data associated with the identified subset of components of the medical device; and
displaying at least a portion of the subset of the log data.

2. The non-transitory computer readable medium of claim 1, wherein the method further comprises:
merging the generated chains to form a sub-chart of the hierarchical components chart;
displaying, on a display device, the sub-chart.

3. The non-transitory computer readable medium of claim 2, wherein the method further comprises:
receiving a selection of one or more nodes of the displayed sub-chart via a user input device; and
displaying the messages of the subset of the log data associated with the component or components corresponding to the selected one or more nodes.

4. The non-transitory computer readable medium of claim 2, wherein the method further comprises:
receiving a selection of one or more nodes of the displayed sub-chart via a user input device; and
updating the subset of the log data by removing the messages of the subset of the log data associated with the component or components corresponding to the selected one or more nodes from the subset.

5. The non-transitory computer readable medium of claim 1, wherein each chain extends upward through the hierarchy of the hierarchical components chart to a root node of the hierarchical components chart representing the medical device.

6. The non-transitory computer readable medium of claim 5, wherein each chain extends upward through the hierarchy of the hierarchical components chart to a highest common node of the hierarchical components chart that is common to all of the generated chains.

7. The non-transitory computer readable medium of claim 2, wherein the method further comprises:
annotating nodes of the displayed sub-chart with messages of the subset of the log data associated with the corresponding components.

8. The non-transitory computer readable medium of claim 7, wherein the method further comprises:
determining whether each message of the subset satisfies a predetermined relevance criterion; and
annotating the nodes of the displayed sub-chart with only those messages that satisfy the predetermined relevance criterion.

9. The non-transitory computer readable medium of claim 1, wherein the medical device comprises a medical imaging device.

10. A non-transitory computer readable medium storing:
instructions readable and executable by at least one electronic processor to perform a method of selecting a subset of log data automatically generated by a medical device undergoing servicing, the method comprising:
receiving, via a user input device, a selection of one or more nodes of a sub-chart displayed on a display device; and
displaying a messages of the subset of the log data associated with the component or components corresponding to the selected one or more nodes.

11. The non-transitory computer readable medium of claim 10, wherein the method further comprises:
receiving a selection of one or more nodes of the displayed sub-chart via a user input device; and
updating the subset of the log data by removing the messages of the subset of the log data associated with the component or components corresponding to the selected one or more nodes from the subset.

12. The non-transitory computer readable medium of claim 10, wherein each chain extends upward through the hierarchy of the hierarchical components chart to a root node of the hierarchical components chart representing the medical device.

13. The non-transitory computer readable medium of claim 12, wherein each chain extends upward through the hierarchy of the hierarchical components chart to a highest common node of the hierarchical components chart that is common to all of the generated chains.

14. The non-transitory computer readable medium of claim 11, wherein the method further comprises:
annotating nodes of the displayed sub-chart with messages of the subset of the log data associated with the corresponding components.

15. The non-transitory computer readable medium of claim 14, wherein the method further comprises:
determining whether each message of the subset satisfies a predetermined relevance criterion; and
annotating the nodes of the displayed sub-chart with only those messages that satisfy the predetermined relevance criterion.

16. The non-transitory computer readable medium of claim 10, wherein the method further comprises:
generating chains of a hierarchical components chart representing components of the medical device, wherein each chain begins at a node of the hierarchical components chart representing a component of the medical device involved in the servicing and extends upward through the hierarchy of the hierarchical components chart;
identifying a subset of components of the medical device corresponding to the nodes of the generated chains;
selecting the subset of the log data as messages of the log data associated with the identified subset of components of the medical device; and
displaying at least a portion of the subset of the log data.

17. The non-transitory computer readable medium of claim 16, wherein the method further comprises:
merging the generated chains to form the sub-chart of the hierarchical components chart;
displaying, on a display device, the sub-chart.

18. The non-transitory computer readable medium of claim 1, wherein the method further comprises:
generating a video showing the subset of messages based on a timestamp of the messages.

19. A method of assisting with servicing of a medical device, the method comprising:
generating chains of a hierarchical components chart representing components of the medical device, wherein each chain begins at a node of the hierarchical components chart representing a component of the medical device involved in the servicing and extends upward through the hierarchy of the hierarchical components chart;
merging the generated chains to form a sub-chart of the hierarchical components chart, wherein the highest node in the hierarchy that is included in the sub-chart is (i) a root node of the hierarchical components chart representing the medical device or (ii) a highest common node of the hierarchical components chart that is common to all of the generated chains; and
displaying, on a display device, the sub-chart.

20. The method of claim 19, wherein each chain extends upward through the hierarchy of the hierarchical components chart to a root node of the hierarchical components chart representing the medical device.
